(19) United States
(12) Patent Application Publication
(10) Pub. No.: US 2013/0281550 A1
(43) Pub. Date: Oct. 24, 2013

(54) CAROTENOID COMPOSITIONS CONTAINING OCTENYL SUCCINATE ANHYDRIDE-MODIFIED GUM ACACIA

(71) Applicants: Andrea Hitzfeld, Basel (CH); Bruno H. Leuenberger, Basel (CH); Olivia Vidoni, Basel (CH)

(72) Inventors: Andrea Hitzfeld, Basel (CH); Bruno H. Leuenberger, Basel (CH); Olivia Vidoni, Basel (CH)

(73) Assignee: DSM IP ASSETS B.V., The Heerlen (NL)

(21) Appl. No.: 13/883,119

(22) PCT Filed: Oct. 4, 2011
(86) PCT No.: PCT/EP2011/067256

§ 371 Date: Jul. 9, 2013

(30) Foreign Application Priority Data
Nov. 3, 2010 (EP) 10189784.1

(51) Int. Cl. A23L 2/58; A61K 47/06; A61K 8/31; A61K 47/36; A61K 8/73

(52) U.S. Cl. CPC A23L 2/58; A61K 47/36; A61K 8/73; A61K 8/31; A61K 47/06
USPC 514/782; 426/540; 426/250

(57) ABSTRACT

The present invention relates to compositions comprising octenyl-succinic anhydride-modified gum acacia and carotenoids. It has been found that the compositions according to the invention allow to produce emulsions having a very high color intensity, and color stability with a low turbidity. These compositions can be used for the enrichment, fortification and/or coloration of food beverages, animal feed, cosmetics or pharmaceutical compositions. The present invention furthermore refers to a process for the manufacture of a beverage by mixing the compositions with ingredients of beverages. The present invention also refers to beverages obtainable by this process.

The present invention relates to compositions comprising octenyl succinate anhydride-modified gum acacia and carotenoids. These compositions can be used for the enrichment, fortification and/or coloration of food beverages, animal feed, cosmetics or pharmaceutical compositions. The present invention also refers to a process for the manufacture of a beverage by mixing the compositions with ingredients of beverages. The present invention further refers to beverages obtainable by this process.

Compositions to enrich, fortify or color food, beverages, animal feed, cosmetics or pharmaceutical compositions which contain carotenoids, for example β-carotene, are known in the art (e.g.: WO 2008110225). β-Carotene is a preferred colorant compound due to its intense and for the above-mentioned applications very pleasing orange color. Since β-carotene is classified as a sparingly soluble in water active and the final products in which it is used are usually aqueous compositions such as beverages, additional compounds have to be added to avoid separation of β-carotene-containing phases in the product, which separation would render the corresponding product unacceptable.

Therefore, carotenoids are often combined with auxiliary compounds such as starches, gums, or fish gelatin, in order to prevent phase separation in a carotenoid containing aqueous composition. Those auxiliary compounds, however, often have a negative influence on the color properties and the nutritional properties of the final products. It is therefore desired to develop new carotenoid compositions, which have high color intensity. Especially, there is need for improved auxiliary compounds, which have very good properties referring to particle size, turbidity, taste, emulsification, emulsion stability, and color in the final product in which it is used.

Gum acacia (also called gum Arabic), a natural hydrocolloid is widely used as an emulsifier/stabilizer in beverage emulsions. It is highly water soluble (up to 50% in weight) and its aqueous solution provides emulsifiability, emulsion stability, encapsulation and film forming ability. Gum acacia is obtained as sticky exudates from the stems and branches of acacia trees when they are subjected to stress. The gum is collected from Acacia senegal trees and, to a lesser extent, from Acacia seyal trees in several countries in the Sahara region of Africa. It is an arabinogalactan polysaccharide with rhamnose and glucoronic acid end units containing two percent proteins (OH-proline, serine, proline) and four sugars (L-arabinose, L-rhamnose, D-galactose, D-glucuronicacid) (Idris et al, Food Hydrocolloids, Part I to III, 12, 1998, 379-388).

However, gum acacia is considered not as good emulsifier as gelatin, an emulsifier commonly used in products, and is not standardized. Uneven performances of the gum may arise among different shipment because of dissimilar functionality related to species, geographical location, and individual growing season. Moreover, the performance of gum acacia depends on the soil, climate, and age of the trees.

In addition to be used as emulsifier, gum acacia is one of the most common carrier materials with carbohydrates such as hydrolyzed starches. In flavor industry, gum arabic is used as fixative in spray dried application where the gum encapsulates the flavor compound, protecting it against oxidation and volatilization. Few works have shown that blends of gum acacia (Krishnan et al., Carbohydrates Polymers, 61, 2005, 95-102; Krishnan et al. Carbohydrates Polymers, 62, 2005, 309-315; Buffo et al, Perfumers & Flavorist, 25, 2000, 45-54), maltodextrin and modified food starches may represent an encapsulating matrix with improved properties regarding flavor retention and protection against oxidation.

In coloring products, such as beverages, it is also often desirable to preserve the optical clarity of the beverage. Sparingly soluble in water colorants, such as carotenoids, e.g. β-carotene, for supplementation are available in many forms, but when added to beverages, will tend to increase the visible turbidity. Ringing, i.e. the formation of a separate fat-soluble β-carotene layer on the top of the liquid, is also a problem of many known β-carotene formulations. One means of adding sparingly soluble substances to beverages without increasing visible turbidity or ringing is to encapsulate the substances in liposomes. However, this is a costly process, and the concentration of substance in the liposome tends to be low.

A satisfactory composition of a sparingly soluble in water colorant, such as a carotenoid, which can be added to beverages in a restorative or nutritionally supplemental amount should thus not affect the optical clarity of the beverage and not alter the sensory properties of the beverage to which it is added. Moreover, the colorant composition should not cause ringing.

WO 2002/069981 discloses a water-soluble esterified hydrocolloid based on dicarboxylic anhydrides and hydrocolloids suitable for producing oil-in-water emulsions characterized in that at least 60% of the oil particles in the emulsion are less than 2 μm in diameter. More specifically, octenyl succinate anhydride modified gum acacia is described as significantly more effective an emulsifier than gum acacia itself. However, WO 2002/069981 only discloses oil-in-water emulsions (typically flavour oils).

Therefore, there is still a need for carotenoid compositions for the enrichment, fortification and/or coloration of food, beverages, animal feed, cosmetics or pharmaceutical compositions which do not show the above-mentioned problems, i.e. which do not show separation phenomena and which provide an increased color intensity and color stability of the resulting product.

It was therefore an object of the present invention to provide carotenoid compositions having an improved color intensity and color stability. These compositions should additionally have the desired properties as indicated above, e.g. very good properties referring to optical clarity and emulsion stability.

It has surprisingly been found that the carotenoid compositions of the present invention comprising an octenyl-succinic anhydride-modified gum acacia and optionally further adjuvants and/or excipients can be mixed with water, whereby the resulting mixture has a high color intensity and color stability. The unexpected high color values have been achieved by using a specific emulsification technology leading to small particle sizes. Such a red to orange color is advantageous for the foods, beverages, animal feed, cosmetic or pharmaceutical compositions the composition can be used for. Further, no separation of the carotenoid from the resulting mixture is obtained. The advantageous color may be achieved without the presence of further auxiliary compounds such as fish gel or coloring compounds beside β-carotene.

The present invention therefore relates to a composition comprising between 5 to 85 weight-% octenyl succinate anhydride modified gum acacia, preferably between 25 to 85 weight-%, most preferably between 55 to 85 and between 0.1 to 50 weight-% of at least one carotenoid wherein, the weight-% is based on the total composition in dry matter.

In a preferred embodiment of the invention, the composition comprises between 0.1 and 30 weight-%, further preferred between 0.2 and 15 weight-%, most preferred between 0.5 and 10 weight-%, and even most preferred between 1 and 5 weight-% carotenoids, based on the total composition in dry matter.

In the most preferred embodiment of the invention, the composition comprises between 55 to 85 weight-% octenyl-succinic anhydride-modified gum acacia and between 1 to 6 weight-% of at least one carotenoid wherein, the weight-% is based on the total composition in dry matter and wherein the carotenoid is selected from the group consisting of beta-carotene and astaxanthin.

Preferably, the compositions of the present invention do not contain further coloring substances except the at least carotenoid compound. Preferably, the compositions of the present invention do not contain fish gelatin.

The octenyl succinate anhydride modified gum acacia can be produced by the person skilled in the art according to WO 2002/069981. Most preferred octenyl succinate anhydride modified gum acacia according to the present invention can be purchased from TIC Gums, Inc., Belcamp, (Md.) USA under the trade name TICAMULSION® A-2010 Powder.

In another preferred embodiment of the invention, the composition is characterized in that the carotenoids are selected from the group consisting of apocarotenal, lutein, bixin, astaxanthin, lycopene, β-carotene, canthaxanthin, citranaxanthin, zeaxanthin, cryptoxanthin, beta-apo-8′-carotenal, and beta-apo12′-carotenal. More preferably, at least one carotenoid is β-carotene or astaxanthin, most preferably, at least one carotenoid is β-carotene.

In an further preferred embodiment of the invention, the composition is characterized in that the composition further comprises one or more adjuvants and/ or excipients, wherein it is preferred that these adjuvants and/or excipients do not represent an oil.

Preferred excipients and/or adjuvants are selected from the group consisting of monosaccharides, disaccharides, oligosaccharides and polysaccharides, water-soluble antioxidants and fat-soluble antioxidants.

Examples of mono- and disaccharides which may be present in the compositions of the present invention are sucrose, invert sugar, xylose, glucose, fructose, lactose, maltose, saccharose and sugar alcohols.

Examples of the oligo- and polysaccharides are starch, starch hydrolysates, e.g. dextrins and maltodextrins, especially those having the range of 5 to 65 dextrose equivalents (DE), and glucose syrup, especially such having the range of 20 to 95 DE. The term “dextrose equivalent” (DE) denotes the degree of hydrolysis and is a measure of the amount of reducing sugar calculated as D-glucose based on dry weight; the scale is based on native starch having a DE close to 0 and glucose having a DE of 100.

The composition according to the invention preferably comprises less than 30% of one or more oil, further preferred less than 20 weight-%, further preferred less than 10 weight-%, further preferred less than 5 weight-%, based on the total composition in dry matter. Most preferably the composition does not comprise any oil.

The expression “oil” as used in this context comprises any trigylcerides or any other oil which is suitable for the desired use of the composition. The triglyceride is suitably a vegetable oil or fat, preferably corn oil, sunflower oil, soybean oil, safflower oil, rapeseed oil, peanut oil, palm oil, palm kernel oil, cotton seed oil, orange oil, limonene, olive oil or coconut oil.

Solid compositions may in addition contain one or more anti-caking agent, such as silicic acid or tricalcium phosphate and the like, and up to 10 weight-%, preferably 0.1 to 5 weight-%.

The water-soluble antioxidant may be for example ascorbic acid or a salt thereof, preferably sodium ascorbate, water-soluble polyphenols such as hydroxy tyrocol and oleuropein, aglycon, epigallo catechin gallate (EGCG) or extracts of rosemary or olives.

The fat-soluble antioxidant may be for example a tocopherol, e.g. dl-α-tocopherol (i.e. synthetic tocopherol), d-α-tocopherol (i.e. natural tocopherol), β- or γ-tocopherol, or a mixture of two or more of these; butylated hydroxytoluene (BHT); butylated hydroxyanisole (BHA); ethoxyquin, propyl gallate; tert. butyl hydroxyquinoline; or 6-ethoxy-1,2-dihydroxy-2,2,4-trimethylquinoline (EMQ), or an ascorbic acid ester of a fatty acid, preferably ascorbyl palmitate or stearate.

Additionally, the composition can further comprise water or any other solvents. If the composition is liquid, it can contain solvents (e.g. water) from few ppm to higher concentration.

Table 1 shows the preferred amounts (weight-%) of ingredients of the composition, based on the total composition in dry matter. The amounts specified in table 1 can additionally be combined with the preferred amounts for the ingredients as specified above.

TABLE 1

| Ingredient | Amount |
|---|---|
| Carotenoids, preferably β- | 0.1 to 50 weight-%, preferably 0.1 to 30, |
| carotene | weight-% preferably 0.5 to 10 weight-%, |
| | most preferably 1 to 5 weight-% |
| octenyl-succinic anhydride- | 5 to 85 weight-%, preferably 25 to 85 |
| modified gum acacia | weight-%; more preferably, 55 to 85 |
| | weight-% |
| saccharides, preferably | 0 to 20 weight-% |
| maltodextrin | |
| a starch hydrolysate | 0 to 20 weight-% |
| oil | 0 to 40 weight-% |
| glycerol | 0 to 30 weight-% |
| a triglyceride | 0 to 30 weight-% |
| one or more water-soluble | 0 to 5 weight-%, preferably 0 to 2 weight-% |
| antioxidant(s) | |
| one or more fat-soluble | 0 to 5 weight-%, preferably 0 to 2 weight-% |
| antioxidant(s) | |
| a starch | 0 to 20 weight-% |
| anti-caking agent | 0.1 to 5 weight-% |

The composition according the present invention can be manufactured by methods known in the art. As an example, the manufacture of the composition comprises the following steps:
I) dissolving octenyl succinate anhydride modified gum acacia in water under stirring at room temperature for at least 1 hour,
II) adding the organic phase, comprising at least one carotenoid and optionally at least one organic solvent, to the solution of step I)
III) homogenizing the mixture of step II) with a conventional emulsification process known to the person skilled in the art,
IV) evaporating the organic solvent under reduced pressure,
V) drying the emulsion by spray-drying, powder catch or other processes.

Therefore, in another embodiment, the composition according to the present invention is characterized in that it is an emulsion. An emulsion is a mixture of two or more immiscible (unblendable) liquids. Emulsions are part of a more general class of two-phase systems of matter called colloids. Although the terms colloid and emulsion are sometimes used interchangeably, emulsion tends to imply that both the dispersed and the continuous phase are liquid. In an emulsion, one liquid (the dispersed phase) is dispersed in the other (the continuous phase).

The compositions of the present invention are preferably additive compositions and are preferably used as additive compositions.

Surprisingly, the particle size of the emulsion with a composition according to the present invention is very small in comparison to standard gum acacia based emulsions, and in comparison to oil-in-water emulsions described in WO 02/069981. Therefore, the present invention is also directed to the use of a composition according to the present invention, as described above, for the enrichment, fortification and/or coloration of food, beverages, animal feed, cosmetics or pharmaceutical compositions, preferably for the enrichment, fortification and/or coloration of beverages wherein, at least 50%, preferably, at least 70%, more preferably, at least 80%, most preferably at least 90%, even more preferably at least 95% of the emulsion particles have a diameter of less than 1 micrometer. Particle size can be measured by methods know in the art e.g.: Coulter Counter.

In another embodiment, the present invention is directed to the use of an emulsion according to the present invention for the enrichment, fortification and/or coloration of beverages, wherein, the nephelometric value of a beverage containing said emulsion in a concentration of 5 ppm is below 20 NTU. NTU stands for Nephelometric Turbidity Units and can be measured by the person skilled in the art from a calibrated nephelometer.

In a further embodiment, the present invention is directed to the use of an emulsion according to the present invention for the enrichment, fortification and/or coloration of beverages, wherein,

a 5 ppm emulsion possesses a color intensity value (E1/1) of at least 800, preferably at least 1000, most preferably at least 1200, even more preferably 1400, wherein the color intensity E1/1 is the absorbance of a 1% solution of the composition and a thickness of 1 cm and is calculated as follows: E1/1=(Amax−A650)*dilution factor/(weight of sample*content of product form in %).

Determination of the color intensity value of the emulsions, which correspond to the emulsification properties, is performed by measuring the absorbance of the compositions. The color intensity values of the emulsions according to the present invention are determined by using a model system containing 5% β-carotene.

To measure the extinction coefficient an adequate amount of the composition according to the present invention is dispersed, dissolved and/or diluted in/with water. The resulting “solution” is diluted to a final concentration of the β-carotene of 5 ppm and its UV/VIS-spectrum is measured against water as reference. From the resulting UV/VIS spectrum the absorbance at the specified wavelength of maximum or shoulder, Amax, is determined. Furthermore, the absorbance at 650 nm, A650, is determined. The color intensity E1/1 is the absorbance of a 1% solution and a thickness of 1 cm and is calculated as follows: E1/1=(Amax−A650)*dilution factor/(weight of sample*content of product form in %).

Other aspects of the invention are food, beverages, animal feed, cosmetics and pharmaceutical compositions containing a composition as described above.

Beverages wherein the product forms of the present invention can be used for enrichment, fortification and/or coloration of beverages can be carbonated beverages e.g., flavored seltzer waters, soft drinks or mineral drinks, as well as non-carbonated beverages e.g. flavored waters, fruit juices, fruit punches and concentrated forms of these beverages. They may be based on natural fruit or vegetable juices or on artificial flavors. Also included are alcoholic beverages and instant beverage powders. Besides, sugar containing beverages diet beverages with non-caloric and artificial sweeteners are also included.

Further, dairy products, obtained from natural sources or synthetic, are within the scope of the food products wherein the product forms of the present invention can be used for enrichment, fortification and/or coloration of the products. Typical examples of such products are milk drinks, ice cream, cheese, yogurt and the like. Milk replacing products such as soymilk drinks and tofu products are also comprised within this range of application.

Also included are sweets which contain the product forms of the present invention for enrichment, fortification and/or coloration of the products, such as confectionery products, candies, gums, desserts, e.g. ice cream, jellies, puddings, instant pudding powders and the like.

Also included are cereals, snacks, cookies, pasta, soups and sauces, mayonnaise, salad dressings and the like which contain the product forms of the present invention for enrichment, fortification and/or coloration of the products. Furthermore, fruit preparations used for dairy and cereals are also included.

The final concentration of the β-carotene which is added via the compositions of the present invention to a food product may preferably be from 0.1 to 50 ppm, particularly from 1 to 30 ppm, more preferred 3 to 20 ppm, e.g. about 6 ppm, based on the total weight of the food composition and depending on the particular food product to be colored or fortified and the intended grade of coloration or fortification.

The food compositions of this invention are preferably obtained by adding to a food product the carotenoid in the form of a composition of this invention. For enrichment, fortification and/or coloration of a food or a pharmaceutical product a composition of this invention can be used according to methods per se known for the application of water dispersible solid product forms.

In general the composition may be added either as an aqueous stock solution, a dry powder mix or a pre-blend with other suitable food ingredients according to the specific application. Mixing can be done e.g. using a dry powder blender, a low shear mixer, a high-pressure homogenizer or a high shear mixer depending on the formulation of the final application. As will be readily apparent such technicalities are within the skill of the expert.

Pharmaceutical compositions such as tablets or capsules wherein the compositions of the present invention are used as a colorant are also within the scope of the present invention. The coloration of tablets can be accomplished by adding the compositions in form of a liquid or solid colorant composition separately to the tablet coating mixture or by adding the compositions to one of the components of the tablet coating mixture. Colored hard or soft-shell capsules can be prepared by incorporating the compositions in the aqueous solution of the capsule mass.

Pharmaceutical compositions such as tablets such as chewable tablets, effervescent tablets or film-coated tablets or capsules such as hard shell capsules wherein the compositions of the present invention are used as an active ingredient are also within the scope of the present invention. The product forms are typically added as powders to the tabletting mixture or filled into the capsules in a manner per se known for the production of capsules.

Animal feed products such as premixes of nutritional ingredients, compound feeds, milk replacers, liquid diets or feed preparations wherein the compositions are either used as a colorant for pigmentation e.g. for egg yolks, table poultry, broilers or aquatic animals or as an active ingredient are also within the scope of the present invention.

Cosmetics, toiletries and derma products i.e. skin and hair care products such as creams, lotions, baths, lipsticks, shampoos, conditioners, sprays or gels wherein the compositions of the present invention are used as a colorant or as an additive or as an active ingredient are also within the scope of the present invention.

The invention also relates to a process for the manufacture of a beverage comprising the steps of homogenizing the composition according to any of claims 1 to 5, and mixing 1 to 50 ppm based on the carotenoid content, preferably 5 ppm, of the emulsified composition with further usual ingredients of beverages.

Further, the present invention relates to beverages obtainable by the process for the manufacture of a beverage as described above.

The present invention is further illustrated by the following examples, which are not intended to be limiting.

### EXAMPLES

### Example 1

### Preparation of Composition Comprising octenyl-succinic anhydride-modified gum acacia and beta-carotene

In a reactor vessel, octenyl-succinic anhydride-modified gum acacia (Ticamulsion® A-2010) was dissolved in deionized water at room temperature with an anchor stirrer until total dissolution. Then the organic phase was added to the aqueous phase and the resulting mixture homogenized. Finally the solvent was evaporated under reduced pressure and the resulting product was converted into a dried form.

| | Amount |
|---|---|
| Composition | (wt %) |
| octenyl-succinic anhydride-modified gum acacia | 75 |
| crystalline β-carotene | 6 |
| d-L-α-tocopherol | 2 |
| corn oil | 7 |
| water | 10 |

### Example 2

### Characterization of Liquid Emulsions

The liquid emulsion of example 1 was characterized via Zeta-potential measurement. Before analysis, this material was diluted in milli-Q water in order to get a minimum concentration of 0.1 to 1% weight in volume required to obtain a sufficient scattering for the measurement. Because such measurement is not size dependant, the Smoluchowski theory was selected for the target calculation. According to this methodology, this product shows a Zeta-potential value of −51.5 mV.

Particle size analyses were performed with dynamic light scattering. The emulsion was diluted with milli-Q water until a concentration below 0.1% by volume and then measured at two angles: 173° and 90° . Particle size obtained is 247 nm at 173° and 226 at 90°.

Laser diffraction was additionally used to determine particle size. In this case, the emulsion was diluted directly in the measuring cell to a suitable obscuration of 20% and the Sauter diameter taken as target value. All the particles are under 1 μm and the Sauter diameter is 270 nm.

For color intensity measurement, a 5 ppm beta-carotene solution was performed and analyzed using a UV/Vis spectrometer. Out of the maximum absorption (Aₘₐₓ) and the absorption at 650 nm (A₆₅₀), color intensity was calculated through the following equation:

((Aₘₐₓ−A₆₅₀)*dilution factor)/(weight sample*content of product form in %).

The resulting value is 1549.

The same solution (5 ppm beta-carotene) was used to determine cloudiness of the material (Turbidity). The nephelometric value of the corresponding product is 13.6 NTU.

### Example 3

### Spray Drying of Liquid Compositions

300 g of β-carotene emulsion as prepared in example 1 was spray-dried under nitrogen in an Mini Spray Dryer B-290 Advanced (Buchi) equipped with a two fluid nozzle, and a high performance cyclone. Emulsion was warmed up to 40° C.; and the process parameters were set to: T°ᵢₙ: 190° C.; T°ₒᵤₜ: 85° C., Flow: 40 m³.hr⁻¹; aspirator: 90%.

40 g of product were collected and stored under nitrogen for further characterization.

### Example 4

### Characterisation of Spray Dried Forms

The spray dried product prepared in example 3 was characterized via Zeta-potential measurement. Before analysis, this material was dissolved in milli-Q water at minimum concentration of 0.1 to 1% weight in volume in order to obtain the sufficient scattering. Because such measurement is not size dependant, the Smoluchowski theory was selected for the target calculation. According to this methodology, this product shows a Zeta-potential value of −60 mV.

Particle size analyses were performed with dynamic light scattering. The spray-dried product was dissolved in milli-Q water until a concentration below 0.1% by volume and then measured at two angles: 173° and 90°. Particle size obtained is 203 nm at 173° and 213 nm at 90°.

Laser diffraction was additionally used to determine particle size. In this case, the dissolved spray dried product was diluted directly in the measuring cell to a suitable obscuration of 20% and the Sauter diameter taken as target value. All the particles are under 1 μm and the Sauter diameter is 270 nm.

For color intensity measurement, a corresponding 5 ppm beta-carotene solution was performed and analyzed using a UV/Ms spectrometer. Out of the maximum absorption (Aₘₐₓ) and the absorption at 650 nm (A₆₅₀), color intensity was calculated through the following equation:

((Aₘₐₓ−A₆₅₀)*dilution factor)/(weight sample*content of product form in %).

The resulting value is 1400.

The same solution (5 ppm beta-carotene) was used to determine cloudiness of the material (Turbidity). The nephelometric value of the corresponding product is 14 NTU.

### Example 5

### Preparation of Composition Comprising octenyl-succinic anhydride-modified gum acacia and astaxanthin

In a reactor vessel octenyl-succinic anhydride-modified gum acacia (Ticamulsion® A-2010) is dissolved in deionized water at room temperature with an anchor stirrer until total dissolution. Then, a solution of astaxanthin, beeswax and dl-alpha-Tocopherol in a solvent is added to the aqueous phase and the resulting mixture homogenized. Finally the solvent is evaporated under reduced pressure and the resulting product spray through a nozzle. The resulting powder is treated with corn starch, and dried.

| | Amount |
|---|---|
| Composition | (wt-%) |
| octenyl-succinic anhydride-modified gum acacia | 67 |
| Corn starch | 18 |
| Astaxanthin | 5 |
| d-L-α-tocopherol | 5 |
| Beeswax | 5 |

1. Composition comprising between 5 to 85 weight-% octenyl-succinic anhydride-modified gum acacia and between 0.1 to 50 weight-% of at least one carotenoid wherein, the weight-% is based on the total composition in dry matter.

2. Composition according to claim 1, characterized in that the composition comprises between 0.5 to 10 weight-% of at least one carotenoid, based on the total composition in dry matter.

3. Composition according to claim 1, characterized in that at least one carotenoid is selected from the group consisting of apocarotenal, lutein, astaxanthin, lycopene, β-carotene, canthaxanthin, citranaxanthin.

4. Composition according to claim 1, characterized in that at least one carotenoid is β-carotene.

5. Composition according to claim 1, characterized in that the composition further comprises one or more adjuvants and/or excipients.

6. Composition according to claim 1, characterized in that the composition is an emulsion.

7. Use of a composition according to claim 6 for the enrichment, fortification and/or coloration of food, beverages, cosmetics or pharmaceutical compositions wherein, at least 50% of the emulsion particles have a diameter of less than 1 micrometer.

8. Use of a composition as in claim 6, wherein at least 90% of the emulsion particles have a diameter of less than 1 micrometer.

9. Use of a composition as in claim 6 wherein, the nephelometric value of the corresponding product is below 20 NTU

10. Use of a composition as in claim 6 wherein, the emulsion possesses a color intensity value (E1/1) of at least 800, wherein the color intensity E1/1 is the absorbance of a 1% solution and a thickness of 1 cm and is calculated as follows: E1/1=(Amax−A650)*dilution factor/(weight of sample*content of product form in %).

11. Process for the manufacture of a beverage comprising the steps of homogenizing the composition according to claim 1, and mixing the emulsified composition with further usual ingredients of beverages.

12. Beverage obtainable by the process according to claim 11.
